(19) United States
(12) Patent Application Publication
(10) Pub. No.: US 2014/0356313 A1
(43) Pub. Date: Dec. 4, 2014

(54) DERMAL PROTECTOR

(71) Applicant: Marie H. JHIN, (US)

(72) Inventors: Marie H. JHIN, Palo Alto, CA (US); Hanafi TANOJO, Palo Alto, CA (US)

(73) Assignee: Marie H. JHIN, Palo Alto, CA (US)

(21) Appl. No.: 14/462,506

(22) Filed: Aug. 18, 2014

Related U.S. Application Data
(63) Continuation of application No. 13/735,687, filed on Jan. 7, 2013, now abandoned.

(51) Int. Cl. A61K 31/78; A61K 31/09; B65B 1/04; A61K 31/155

(52) U.S. Cl. CPC A61K 31/78; A61K 31/155; A61K 31/09; B65B 1/04
USPC 424/78.07; 53/473

(57) ABSTRACT

A dermal protector liquid including a barrier and antimicrobial materials is disclosed. The protector liquid can be sprayed onto the skin. The protector liquid can have a dispersant and moisturizer. When the base and solvents of the liquid evaporate, a polymer barrier and antimicrobial material can continue to reside on the skin.

### CROSS-REFERENCE TO RELATED APPLICATIONS

This application is a continuation of U.S. patent application Ser. No. 13/735,687, filed on Jan. 7, 2013, the content of which is incorporated herein by reference in its entirety.

### BACKGROUND

Reducing exposure to infection through the skin is critical in minimizing the spread of many diseases. In many environments, it is difficult if not impossible to prevent contact between one's skin and a potentially infectious surface, such as shaking hands, touching a door handle, or making contact with exercise mats.

Antimicrobial gels or lotions are available for dermal application. However, they quickly evaporate resulting in only short-term effectiveness. Accordingly, repeated application of the gel in a short period of time is necessary to have any level of sustained effectiveness. Furthermore, the gels only act in an antimicrobial fashion, but do not provide a barrier to initial transmission of infectious agents.

Also, the application of gels requires rubbing the dermis. This rubbing can irritate sensitive skin and obviously contaminates another surface (often more dermis, such as on the opposite hand) with the gel whether desired or not. Also, the gel is frequently applied unevenly and requires extended rubbing for even and adequate application over a large area of dermis.

Also, effective barrier layers for the skin are often available in the form of clothing including, gloves, bandages and gauze. These dermis barrier layers are often loose, and inconvenient, to wear in some situations, such as gloves and full-body clothing during exercise, such as stretching or yoga on a mat. Clothing, providing complete, contiguous coverage, such as complete coverage “bunny suits”, are not light, convenient, nor skin tight, and are typically bulky, inconvenient and socially unacceptable for regular use.

The antimicrobial gels only offer a short-term barrier, if that, until the gel is rubbed off.

Accordingly, a dermal protector is desired that can provide sustained effectiveness against the transmission of infectious microbes. A dermal protector that can act as a long-term barrier and has anti-microbial properties is desired. Furthermore, a dermal protector that can be applied without rubbing the skin is desired.

### SUMMARY OF THE INVENTION

A dermal protector is disclosed herein. The dermal protector can be a material that can be stored in a liquid form and can be applied to the skin and dry into an antimicrobial barrier layer.

The dermal protector can have an antimicrobial solution, a barrier material, a wetting agent, a carrier material, and a dispersant agent. The dermal protector can also have a moisturizer, gelatinous materials, one or more fragrances and coloring agents, or combinations thereof.

The antimicrobial solution can have a solvent, wherein between 0.5% and 4% of the protector liquid is the antimicrobial solution. The antimicrobial material can include chlorhexidine, triclosan, ethanol, or combinations thereof.

0.25% to 1.5%, for example 0.75% of the protector can be the barrier material. The barrier material can include Eudragit RLPO. The barrier material can be water-repellent and/or water-resistant.

The wetting agent can include alcohol, such as ethanol. The carrier can be water. The dispersant can be propylene glycol.

A method of protecting the dermis is disclosed. The method can include spraying a liquid onto the dermis and drying the liquid on the dermis. The liquid can include an antimicrobial solution, a barrier material; a wetting agent, a carrier material, and a dispersant agent. The antimicrobial solution can have an antimicrobial material and a solvent. Between 0.5% and 4% of the protector liquid can be the antimicrobial solution. Drying can include evaporating the carrier material, the dispersant agent, and the wetting agent.

A method of making a dermal protection liquid is disclosed. The method includes mixing an antimicrobial solution, a barrier material, a wetting agent, a carrier material; and a dispersant agent into the dermal protection liquid. The method further includes packaging the dermal protection liquid in a bottle comprising a spray dispenser.

### DETAILED DESCRIPTION

A dermal protector is disclosed that can have one or more antimicrobial ingredient, one or more barrier ingredient, one or more solvents and/or wetting agents, one or more carriers (e.g., water), dispersant agents, and combinations thereof. The dermal protector can be applied topically to the skin. The dermal protector can be in a liquid state during storage. The dermal protector can be applied by spraying, brushing, dipping or rubbing onto the skin.

The dermal protector can be an antiseptic non-irritant topical formulation of an aqueous composition. The dermal protector can act as an antimicrobial agent and a barrier on the skin. The antimicrobial ingredient can be a material capable of producing an inhibition to the microbial growth, including bacteria, yeast, fungi, or virus. The quantitative assessment of microbial growth inhibition or antiseptic efficacy can be defined by the rate of reduction of microbial organism number as can be calculated by the formula:

(l/t)(log (Nₒ/N))

in which t is the time, in minutes, for the microbial count to be reduced from Nₒ to N microbes; where Nₒ is the initial number of organisms, in cfu per mL; and N is the final number of organisms, in cfu per mL, of organisms. Using the formula shown above, the antiseptic efficacy can be from about 15 to about 70, more narrowly from about 21 to about 68, yet more narrowly from about 24 to about 60. For example, the log component (i.e., log (Nₒ/N)) can be from about 1.75 to about 2.25, for example about 2, over a time of about 2 to about 5 seconds.

The antimicrobial ingredient can be chlorhexidine, chloroxylenol, hexachlorophene, iodine and iodophors, quaternary ammonium compounds, triclosan, alcohol such as ethanol, or combinations thereof.

The antimicrobial ingredient can be a solution, such as one or more of the antimicrobial ingredients disclosed, supra, in water, for example about a 20% solution in water.

The antimicrobial ingredient can be from about 0.5% to about 5%, more narrowly from about 1%, to about 3%, for example about 2% of the dermal protector.

The barrier ingredient can be formed by deposition of a thin impermeable or semi-permeable membrane on the surface of the skin. The harder ingredient can be impermeable (e.g., due to the nature of polymer) independent of the thickness, or semi-permeable, (e.g., due to the lattice structure) but the permeability may be independent of pore size or thickness when dry. Barrier impermeability can be quantified by Transepidermal Water Loss (TEWL) measurements. TEWL measures the water vapor rate exiting the skin surface. Normal TEWL value of about 5-10 g/m²/h in the forearm of a healthy adult can be affected by the application of an impermeable or semipermeable barrier. For example, barriers can reduce TEWL below about 5 g/m²/h more narrowly to less than about 2.5 g/m²/h, yet more narrowly to less than about 1 g/m²h.

The harder ingredient can be a copolymer of ethyl acrylate, methyl methacrylate and a low content of methacrylic acid ester with quaternary ammonium groups (e.g., Eudragite® PO from Evonik Industries of Essen, Germany), acrylate polymer or copolymer; carbomer; polyvinylpyrrolidone: aminoalkyl methacrylate copolymer, or combinations thereof. For example, the barrier ingredient can have polyethyl acrylate, polymethyl methacrylate, polytrimethylammonioethyl methacrylate chloride (or combinations thereof) in a ratio of 1:2:0.2, respectively.

The barrier ingredient can be from about 0.25% to about 1.5%, more narrowly from about 0.5% to about 1.0%, for example about 0.75% of the dermal protector.

The solvent and/or wetting agent can be an ingredient that can dissolve the harrier ingredient and/or other solid ingredients into a solution. The solvent and/or wetting agent can be alcohol such as ethanol, isopropanol, or combinations thereof.

The solvent ingredient can be from about 10% to about 30%, more narrowly from about 15% to about 25%, for example about 21.5% of the dermal protector.

The dispersant agent can be a material to create an even coating of the dermis by the dermal protector when the dermal protector is applied to the dermis. The dispersant agent can be propylene glycol or hexylene glycol. The solvent can act as the dispersant agent.

The dispersant agent can be from about 1% to about 10%, for example about 5.2% of the dermal protector.

The dermal protector can have a moisturizing agent to moisturize the epidermis, preventing dry skin. The moisturizing agent can have occlusive (e.g., dimethicone, petrolatum, paraffin, lanolin), humectant (e.g., glycerin, sorbitol, sodium hyaluronate, urea, propylene glycol, alpha hydroxy acids, sugars, glucosamines, amino acids) and/or emollient (e.g., lanolin, mineral oil, and petrolatum) ingredients, or combinations thereof.

The moisturizing agent can also be the dispersant agent, for example propylene glycol. The moisturizing agent can be from about 1% to about 10%, for example about 5.2% (e.g., when the moisturizing agent and the dispersant agent are the same material, such as propylene glycol) of the dermal protector.

The dermal protector can be transparent or clear, or can be translucent or opaque, for example having coloring agents such as annatto, bismuth oxycholoride, bronze powder, caramel, carmine, beta-carotene, copper powder, guanine, iron oxide, manganese violet, mica, titanium dioxide, zinc oxide, D&C black, blue, brown, green, orange, red, violet, yellow, and FD&C blue, green, red, and yellow. The coloring agent can be less than or equal to about 0.5%, more narrowly less than or equal to about 0.1%, or can be 0% of the dermal protector.

The dermal protector can have fragrances such as oils (e.g., agarwood, cedarwood, sandalwood, rosemary, orange, lime, pennyroyal, currant, cucumber) vanilla tincture, oil, extract, oleoresin, methyl vanillate, bergamot, oakmoss, patchouli, or combinations thereof. The fragrance can be less than or equal to about 0.5%, more narrowly less than or equal to about 0.1%, or can be 0% of the dermal protector.

The carrier, vehicle, or substrate fluid can be water, deionized water, or a combination thereof. The carrier can be measured separately from water or other carriers present in the other ingredient solutions. The carrier can be from about 50% to about 90% of the dermal protector, for example about 70% or 70.05%.

The dermal protector can have one or more UV-blocking ingredients, such as triazine derivatives, benzophenone or mixture thereof at the levels as required to meet the protective strength listed in the product label of the UV-blocking ingredient.

The dermal protector can have a pH from about 5.0 to about 8.5, for example about 6.0. The dermal protector can have stabilizers or buffers, such as citric acid and its salts, phosphoric acid and its salts, or combinations thereof. The stabilizer or buffer can be less than or equal to about 2% of the dermal protector, more narrowly less than or equal to about 1% of the dermal protector, for example about 0%.

The dermal protector can be a homogeneous liquid before application to the dermis. The dermal protector can have a viscosity from about 0.5 cP to about 2.0 cP, for example about 1.02 cP at 20°C.. Gelatinous materials, such as gelatin, can be added to the formulations described herein so the dermal protector can be a gel.

After application to the skin and drying, the dermal protector can leave a water-repellent or water-insoluble residue film barrier on the dermis. The dermal protector and barrier can be non-irritants to the skin.

### EXAMPLE

5.2 parts (by weight) propylene glycol, 21.5 parts ethanol, 70.05 parts deionized water and 2.5 parts 20% chlorhexidine in water solution were weighed into a mixer. This formulation was mixed until the solids were visually dissolved. 0.75 parts of a mix of copolymer of ethyl acrylate, methyl methacrylate and a low content of methacrylic acid ester with quaternary ammonium groups (e.g., Eudragit RLPO) was slowly added to the formulation under constant mixing. The formulation was then left to mix until visually homogenous, resulting in a variation of the dermal protector.

30 mL (1 fl. Oz) of the dermal protector can be packaged in a dark colored glass or plastic bottle. The bottle can have an atomizer spray pump, open top, pump dispenser, brush, sponge, or eyedropper applicator, or combinations thereof. The package can be stored in a cool and dry environment.

The dermal protector can be wiped, spread, sprayed (e.g., the applicator does not have to touch the skin), dropped on the target skin, the target skin can be dipped into the dermal protector, or combinations thereof. The dermal protector can be spread on the skin by the applicator or by the user's hands. The dermal protector can be sprayed onto the skin with a spray droplet size from 200 to 100 microns (e.g., by a fine atomizer) or up to 1000 microns (e.g., a semi-coarse atomizer).

Upon application to the skin, the dermal protector can act as an antimicrobial, After application to the skin, the dermal protector can dry (e.g., water, ethanol, and propylene glycol in the dermal protector can evaporate), leaving a residue barrier remaining on the skin. The residue can act as a harrier and continue to act as an antimicrobial.

The % compositions shown herein are by weight (i.e., “% w/w”)

Any elements described herein as singular can be pluralized (i.e., anything described as “one” can be more than one). Any species element of a genus element can have the characteristics or elements of any other species element of that genus. The above-described materials, methods, and their elements for carrying out the invention, and variations of aspects of the invention can be combined and modified with each other in any combination.

We claim:

1. A dermal protector liquid comprising:
an antimicrobial solution comprising an antimicrobial material and a solvent, wherein between 0.5% and 4% of the protector liquid is the antimicrobial solution;
a barrier material;
a wetting agent;
a carrier material; and
a dispersant agent.

2. The liquid of claim 1, wherein the antimicrobial material comprises chlorhexidine.

3. The liquid of claim 1, wherein the antimicrobial material comprises triclorsan.

4. The liquid of claim 1, wherein 2% of the protector is the antimicrobial solution.

5. The liquid of claim 1, wherein 0.1% to 0.8% of the protector is the antimicrobial material.

6. The liquid of claim 1, wherein 0.5% of the protector is chlorhexidine.

7. The liquid of claim 1, wherein 0.25% to 1.5% of the protector is the barrier material.

8. The liquid of claim 1, wherein 0.75% of the barrier is the barrier material.

9. The liquid of claim 1, wherein the barrier material comprises Eudragit RLPO

10. The liquid of claim 1, wherein the wetting agent comprises alcohol.

11. The liquid of claim 1, wherein the carrier comprises water.

12. The liquid of claim 1, wherein the dispersant comprises propylene glycol.

13. A method of protecting the dermis comprising:
spraying a protector liquid onto the dermis, wherein the protector liquid comprises an antimicrobial solution comprising an antimicrobial material and a solvent, wherein between 0.5% and 4% by weight of the protector liquid is the antimicrobial solution, a barrier material; a wetting agent, a carrier material, and a dispersant agent, drying the protector liquid on the dermis, wherein drying comprises evaporating the carrier material, the dispersant agent, and the wetting agent, creating a barrier, wherein the barrier is water insoluble, wherein the barrier comprises the barrier material, and
preventing contact between the dermis and an infectious surface, wherein the barrier prevents the contact.

14. The method of claim 13, wherein the antimicrobial material comprises chlorhexidine.

15. The method of claim 13, wherein the antimicrobial material comprises triclosan triclorsan.

16. The method of claim 13, wherein the barrier material comprises of a copolymer Eudragit RLPO.

17. The method of claim 13, wherein the wetting agent comprises alcohol.

18. The method of claim 13, wherein herein the carrier material comprises water.

19. The method of claim 13, wherein herein the dispersant agent comprises propylene glycol.

20. A method of making a dermal protection liquid comprising:
mixing an antimicrobial solution comprising an antimicrobial material and a solvent, wherein between 0.5% and 4% of the protector liquid is the antimicrobial solution, a barrier material, a wetting agent, a carrier material; and a dispersant agent, wherein mixing comprises producing the dermal protection liquid; and
packaging the dermal protection liquid in a bottle comprising a spray dispenser.
